(19) United States
(12) Patent Application Publication
(10) Pub. No.: US 2025/0387605 A1
(43) Pub. Date: Dec. 25, 2025

(54) NEEDLELESS CONNECTOR WITH FOLDING SEAL

(71) Applicant: CAREFUSION 303, INC., San Diego, CA (US)

(72) Inventors: Raymond P. FEITH, Chino Hills, CA (US); Gene E. MASON, La Habra Heights, CA (US)

(21) Appl. No.: 19/249,909

(22) Filed: Jun. 25, 2025

Related U.S. Application Data
(60) Provisional application No. 63/664,082, filed on Jun. 25, 2024.

(51) Int. Cl. A61M 39/04; A61M 5/14; A61M 25/00; A61M 39/00; A61M 39/02

(52) U.S. Cl. CPC A61M 39/045; A61M 5/1413; A61M 25/0097; A61M 2039/0081; A61M 2039/0205; A61M 2039/0258; A61M 2039/027; A61M 2207/00

(57) ABSTRACT

Seals for medical fluid connectors are disclosed. A seal for a medical fluid connector may include a first portion and a second portion compressed together to form the seal. The inner surfaces of the first and second portions contain recesses. When the first and second portions are compressed together, fluid cannot pass between the portions. In other words, fluid cannot pass through the seal. The seal is penetrable by a center post at an insertion area compressed by the recesses. When the center post penetrates the seal, the center post provides a fluid path through the seal. The recesses also contain a lubricant that limits the amount of friction between the seal and the center post.

### RELATED APPLICATION DATA

This application claims priority to U.S. Provisional Application No. 63/664,082, filed Jun. 25, 2024, the entire content of which is incorporated herein by reference.

### TECHNICAL FIELD

The present disclosure relates generally to medical fluid connectors and, more particularly, to neutral displacement needle-free connectors that reduce the occurrence of dislodgement of an intravenous (IV) catheter that is connected to a patient.

### BACKGROUND

Medical treatments often include the infusion of a medical fluid (e.g., a saline solution or a liquid medication) to patients using an IV catheter that is connected through an arrangement of flexible tubing and fittings, commonly referred to as an “IV set,” to a source of fluid, for example an IV bag. Often, tubing or catheters are coupled or secured to each other to allow fluid communication between various portions of tubing or catheters.

In some applications, such tubing or catheters may become dislodged due to improper securement and/or when the coupling is subject to forces greater than what the coupling is designed to withstand.

Manufacturing medical fluid connectors and, more particularly, neutral displacement needle-free connectors often requires use of razors to cut slits in the seals of such connectors.

### SUMMARY

In accordance with at least some embodiments disclosed herein is the realization that the process of manufacturing medical fluid connectors—more particularly, the use of razors to cut slits in the seals of such connectors—can result in inconsistent connectors. Additionally, the use of razors to cut slits in the seals of medical fluid connectors can create corners with high stress conditions that are susceptible to tearing. Such tearing can decrease the ability of the connectors to properly seal.

Aspects of the present disclosure provide a medical fluid connector that is manufactured without the use of razors. Molding, compression, and/or lubrication are used to manufacture points of decreased tension that serve the same purpose as the slits in traditional medical fluid connectors.

Accordingly, aspects of the present disclosure provide a seal for a needleless fluid connector, the seal comprising: a first portion comprising a first top end, a first bottom end, and a first surface extending between the first top end and the first bottom end, and a second portion comprising a second top end, a second bottom end, and a second surface extending between the second top end and the second bottom end, the first portion being configured to couple with the second portion such that the first surface contacts the second surface and a longitudinal axis extends along the first and second surfaces from the first and second top ends to the first and second bottom ends; a guiding recess and a lubricating recess on the first surface, the guiding recess extending from the first top end to at least the lubricating recess, and the lubricating recess positioned within the guiding recess and containing a lubricant, wherein coupling of the first portion with the second portion forms an insertion area on the first top end aligned with the guiding recess.

In some embodiments, the insertion area comprises an area of decreased tension that is formed from the guiding recess by compressing the first portion and the second portion together.

In some embodiments, a fluid cannot permeate the insertion area and contact the first and second surfaces.

In some embodiments, the insertion area assists a center post in penetrating the seal, and the center post creates a fluid path through the seal when the center post penetrates the seal.

In some embodiments, the guiding recess is located along the longitudinal axis.

In some embodiments, the guiding recess extends from the first top end to the first bottom end.

In some embodiments, the lubricant in the lubricating recess is configured to lubricate the center post when the center post is inserted past the lubricating recess.

In some embodiments, the second surface comprises a second guiding recess and a second lubricating recess, wherein the second guiding recess extends from the second top end to at least the second lubricating recess, and the second lubricating recess is positioned within the second guiding recess and contains the lubricant.

In some embodiments, the second guiding recess is located along the longitudinal axis.

In some embodiments, the second guiding recess extends from the second top end to the second bottom end.

In some embodiments, the lubricant in the second lubricating recess is configured to lubricate the center post when the center post is inserted past the second lubricating recess.

In some embodiments, a divot is located on at least one of the first and second bottom ends, wherein a path length between the first top end and the divot is less than a length between the first top end and the first bottom end, whereby the divot provides a shortened path of the center post through the seal.

In some embodiments, the first and second surfaces each comprise protrusions and intrusions, and the protrusions and intrusions on the first surface correspond with intrusions and protrusions on the second surface.

In some embodiments, each protrusion mates with its corresponding intrusion when the first portion and the second portion are compressed together.

Aspects of the present disclosure also provide A method for manufacturing a seal for a needleless medical fluid connector, the method comprising: molding a first portion and a second portion; molding a guiding recess on a first surface of the first portion; molding a lubricating recess inside the guiding recess; applying a lubricant to the lubricating recess; engaging the first surface against a second surface of the second portion; and compressing the first and second portions together to form a seal by applying forces perpendicular to the first and second surfaces.

In some embodiments, compressing the first and second portions together comprises preventing fluid from passing between the first and second portions.

In some embodiments, compressing the first and second portions together comprises forming an insertion area that is configured to receive a center post that penetrates the seal and provides a fluid path through the seal.

In some embodiments, applying the lubricant to the lubricating recess assists the center post in penetrating the seal.

In some embodiments, a second guiding recess is molded on the second surface, a second lubricating recess is molded inside the second guiding recess, and the lubricant is applied to the second lubricating recess.

In some embodiments, at least one of the first and second surfaces comprise protrusions and intrusions, and the intrusions on the at least one of the first and second surfaces are configured to receive corresponding protrusions on the other of the at least one of the first and second surfaces, the method further comprising engaging the first surface against the second surface and mating each protrusion to its corresponding intrusion.

Accordingly, the present application addresses several manufacturing and operational challenges encountered in prior medical fluid connectors that are susceptible to manufacturing inconsistencies and premature tearing.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure.

FIG. 2 illustrates a cross-sectional view of the medical fluid connector in FIG. 1, in accordance with aspects of the present disclosure.

FIG. 3 illustrates a perspective view of the seal in a fully assembled position, in accordance with aspects of the present disclosure.

FIG. 4A illustrates a perspective view of the seal in a sub-assembled position, in accordance with aspects of the present disclosure.

FIG. 4B illustrates a perspective view of the seal in a sub-assembled position, in accordance with aspects of the present disclosure.

FIG. 5 illustrates a perspective view of the seal in a pre-assembled position, in accordance with aspects of the present disclosure.

FIG. 6 illustrates a flowchart showing a method for manufacturing a seal for a needleless medical fluid connector, in accordance with aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure includes various features and advantages of manufacturing medical fluid connectors without the use of razors, thus minimizing the likelihood of manufacturing inconsistent connectors and forming corners with high stress conditions that are susceptible to tearing.

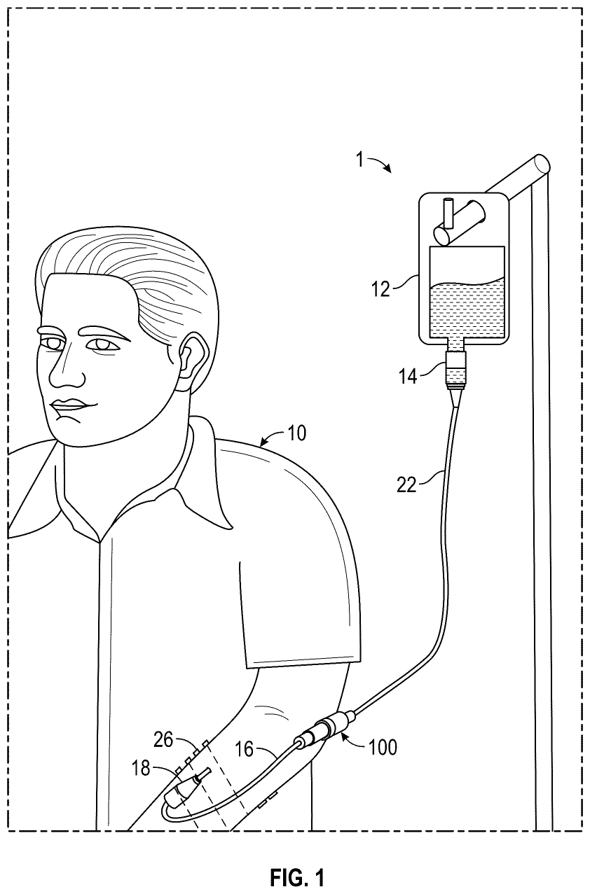

Referring now to the figures, FIG. 1 illustrates an IV set 1 coupled to a patient 10, in accordance with the aspects of the present disclosure. The IV set 1 includes a medical fluid bag 12, a drip chamber 14, and tubing 22. The tubing 22 extends between the drip chamber 14 and a medical fluid connector 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

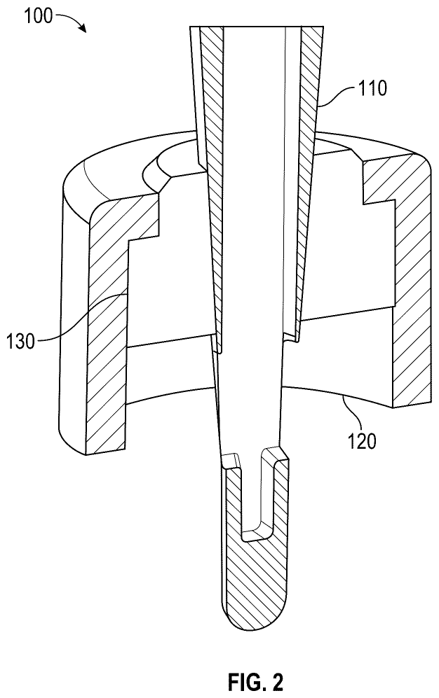

FIG. 2 illustrates a cross-sectional view of the medical fluid connector 100 for use with an IV set, in accordance with the aspects of the present disclosure. The medical fluid connector 100 is designed for use in medical applications, such as the IV set 1 as well as other IV medical fluid delivery applications using catheters, including peripheral intravenous catheters (PIVC), as non-limiting examples.

The medical fluid connector 100 provides a fluid path. As shown, the medical fluid connector 100 includes a center post 110, a seal housing 120, and a seal 130. The center post 110 penetrates the seal 130 to create the fluid path.

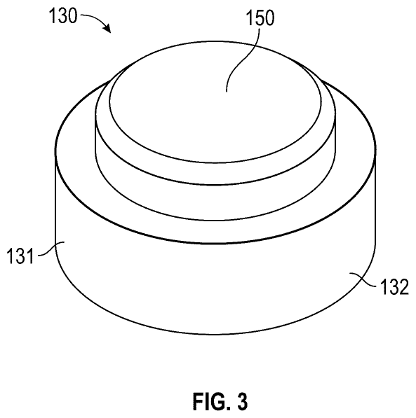

FIG. 3 illustrates a perspective view of the seal 130 in a fully assembled position, in accordance with the aspects of the present disclosure. The seal 130 may include a cylindrical, or generally cylindrical body with a varying diameter. The seal 130 is formed when the first portion 131 and the second portion 132 are compressed together in a mold to form a singular structure or piece. The top of the seal 130 includes an insertion area 150. The insertion area 150 can include a pocket. Fluid cannot permeate the insertion area 150. However, the center post 110 may penetrate the seal 130 and create the fluid path.

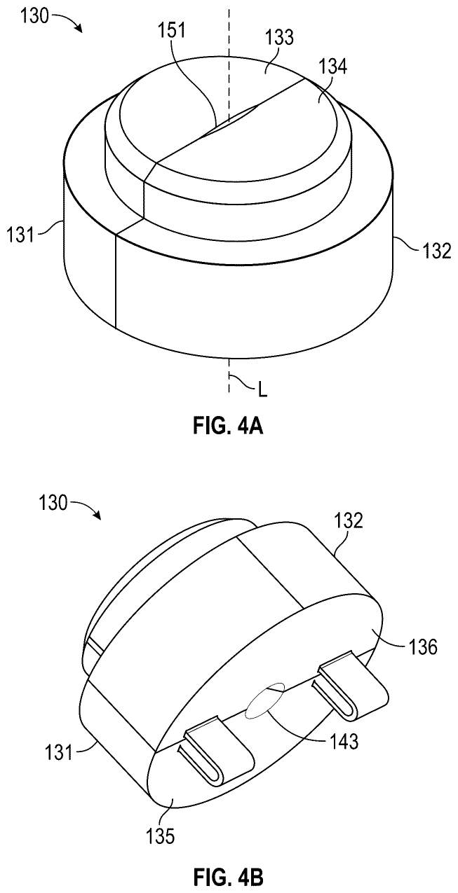

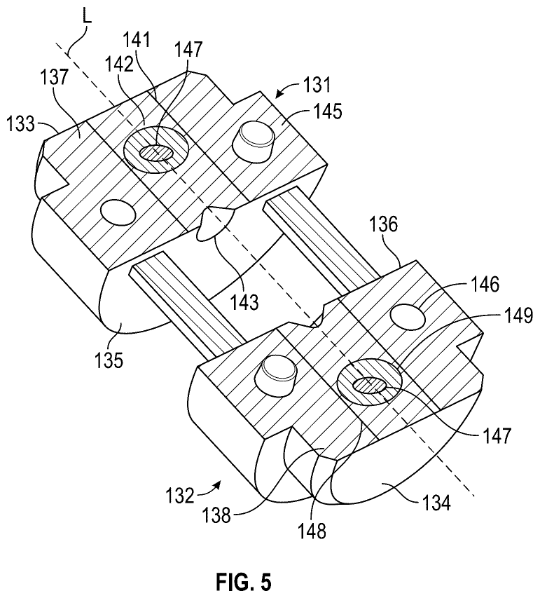

FIGS. 4A and 4B illustrate perspective views of the seal 130 in a sub-assembled position, in accordance with the aspects of the present disclosure. FIG. 4A depicts an embodiment of the seal 130 wherein the first and second portions 131, 132 are halves. The first half 131 has a first top end 133 and a first bottom end 135. The second half 132 has a second top end 134 and a second bottom end 136. The seal 130 becomes fully assembled when the first half 131 of the seal 130 is compressed against a second half 132 of the seal 130. For the seal 130 to become fully assembled, the first and second halves 131, 132 are compressed together by a force that may be perpendicular to the first surface 137 and the second surface 138 (shown in FIG. 5). The insertion area 150 (shown in FIG. 3) is formed when an area of decreased tension 151, which is along the longitudinal axis L in between the first half 131 and the second half 132, is compressed by a force that may be perpendicular to the first surface 137 and the second surface 138 (shown in FIG. 5).

FIG. 5 illustrates a perspective view of the seal 130 in a pre-assembled position, in accordance with the aspects of the present disclosure. Any number of straps may connect the first bottom end 135 to the second bottom end 136. The seal 130 becomes sub-assembled when the straps the seal 130 are folded such that the first surface 137 engages with the second surface 138. The first and second halves 131, 132 may be held together in the sub-assembled position by protrusions 145 on the first and second surfaces 137, 138 mating with the corresponding intrusions 146 on the first and second surfaces 137, 138. The coupling of the protrusions 145 and the intrusions 146 foster consistent assembly of the first and second halves 131, 132. The first and second surfaces 137, 138 may contain any number of protrusions 145. However, there is at least one intrusion 146 for every protrusion 145, and each intrusion 146 is in a location that is symmetrical to the location of a protrusion 145 with respect to the longitudinal axis L.

The first surface 137 may contain a guiding recess 141 along the longitudinal axis L. The guiding recess 141 may extend from the first top end 133 to the first bottom end 135 as shown in FIG. 5. In some embodiments, the guiding recess 141 does not extend this entire length. When the first half 131 and the second half 132 of the seal 130 are compressed together, the guiding recess 141 enables the formation of the insertion area 150.

A lubricating recess 142 may be located along the longitudinal axis L inside the guiding recess 141. The lubricating recess 142 may have a smaller volume than the guiding recess 141. The lubricating recess 142 may contain a lubricant 147 that limits the amount of friction between the seal 130 and the center post 110 when the center post 110 penetrates the seal 130 at the insertion area 150.

The second surface 138 may contain a second guiding recess 148 along the longitudinal axis L. The second guiding recess 148 may extend from the second top end 134 to the second bottom end 136. In some embodiments, the second guiding recess 148 does not extend this entire length.

A second lubricating recess 149 may be located along the longitudinal axis inside the second guiding recess 148. The second lubricating recess 149 may have a smaller volume than the second guiding recess 148. The second lubricating recess 149 may contain a lubricant 147 that limits the amount of friction between the seal 130 and the center post 110 when the center post 110 penetrates the seal 130 at the insertion area 150.

A depression or divot 143 may be located on first bottom end 135 and/or the second bottom end 136 along the longitudinal axis L. The divot 143 may have a generally conical shape with the vertex of the cone pointing towards the top of the seal 130, but other shapes are also possible. A path length between the first top end 133 and the first bottom end 135 is less than a length between the first top end 133 and the top of the divot 143. Likewise, a path length between the second top end 134 and the second bottom end 136 is less than a length between the second top end 134 and the top of the divot 143. That is, the divot 143 can reduce the path length of the center post 110 through the seal 130. Consequently, the friction between the seal 130 and the center post 110 is limited by the presence of the divot 143. In some embodiments, the divot 143 guides the center post 110 as it penetrates the seal 130.

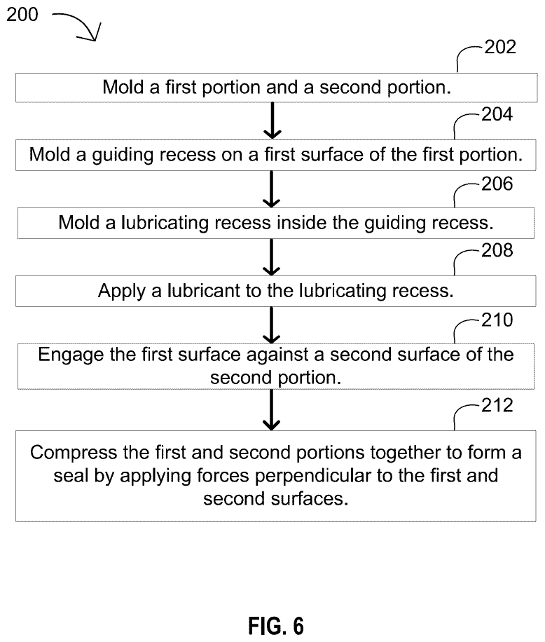

FIG. 6 illustrates a flowchart 200 showing a method for manufacturing a seal for a needleless medical fluid connector, in accordance with aspects of the present disclosure. Seals for needleless medical fluid connectors shown or described herein are manufactured by carrying out the steps of the method shown in the flowchart 200.

In step 202, a first portion and a second portion are molded. Each portion may have an inner face comprising a flat surface. In some embodiments, the first and second portions are first and second halves.

In step 204, a guiding recess is molded on the inner face of the first half. In some embodiments, a second guiding recess is also molded on the inner face of the second half. The guiding recess may extend from the top end of a half to the bottom end of a half. The guiding recess may be located along the center of the inner face of a half.

In step 206, a lubricating recess is molded inside the guiding recess on the inner face of the first half. In some embodiments, a second lubricating recess is also molded inside the second guiding recess on the inner face of the second half. The lubricating recess may have a smaller volume than the first well.

In step 208, a lubricant is applied to the lubricating recess.

In step 210, the inner faces of the first and second halves are engaged against each other. This is a sub-assembled position. The inner face of each half may contain protrusions. The inner face of each half contains an intrusion for every protrusion. In the sub-assembled position, the protrusions are mated with their corresponding intrusions.

In step 212, the first and second halves are compressed together to form a seal by applying forces perpendicular to the inner faces of each half. When the first and second halves are compressed together to form the seal, a fluid cannot pass through the seal. However, the guiding recess form an insertion area on the top end of the seal upon compression, and a center post can penetrate the seal through the insertion area. When the center post penetrates the insertion area, a fluid path is created through the seal through which fluid can pass through the seal.

The lubricant applied to the lubricating recess may limit the amount of friction between the seal and a center post that may penetrate the seal.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A seal for a needleless fluid connector, the seal comprising: a first portion comprising a first top end, a first bottom end, and a first surface extending between the first top end and the first bottom end, and a second portion comprising a second top end, a second bottom end, and a second surface extending between the second top end and the second bottom end, the first portion being configured to couple with the second portion such that the first surface contacts the second surface and a longitudinal axis extends along the first and second surfaces from the first and second top ends to the first and second bottom ends; a guiding recess and a lubricating recess on the first surface, the guiding recess extending from the first top end to at least the lubricating recess, and the lubricating recess positioned within the guiding recess and containing a lubricant, wherein coupling of the first portion with the second portion forms an insertion area on the first top end aligned with the guiding recess.

Clause 2. The seal of clause 1, wherein the insertion area comprises an area of decreased tension that is formed from the guiding recess by compressing the first portion and the second portion together.

Clause 3. The seal of clause 1, wherein a fluid cannot permeate the insertion area and contact the first and second surfaces.

Clause 4. The seal of clause 1, wherein the insertion area assists a center post in penetrating the seal, and the center post creates a fluid path through the seal when the center post penetrates the seal.

Clause 5. The seal of clause 1, wherein the guiding recess is located along the longitudinal axis.

Clause 6. The seal of clause 1, wherein the guiding recess extends from the first top end to the first bottom end.

Clause 7. The seal of clause 1, wherein the lubricant in the lubricating recess is configured to lubricate the center post when the center post is inserted past the lubricating recess.

Clause 8. The seal of clause 1, wherein the second surface comprises a second guiding recess and a second lubricating recess, wherein the second guiding recess extends from the second top end to at least the second lubricating recess, and the second lubricating recess is positioned within the second guiding recess and contains the lubricant.

Clause 9. The seal of clause 8, wherein the second guiding recess is located along the longitudinal axis.

Clause 10. The seal of clause 8, wherein the second guiding recess extends from the second top end to the second bottom end.

Clause 11. The seal of clause 8, wherein the lubricant in the second lubricating recess is configured to lubricate the center post when the center post is inserted past the second lubricating recess.

Clause 12. The seal of clause 1, further comprising a divot located on at least one of the first and second bottom ends, wherein a path length between the first top end and the divot is less than a length between the first top end and the first bottom end, whereby the divot provides a shortened path of the center post through the seal.

Clause 13. The seal of clause 1, wherein the first and second surfaces each comprise protrusions and intrusions, and the protrusions and intrusions on the first surface correspond with intrusions and protrusions on the second surface.

Clause 14. The seal of clause 13, wherein each protrusion mates with its corresponding intrusion when the first portion and the second portion are compressed together.

Clause 15. A method for manufacturing a seal for a needleless medical fluid connector, the method comprising: molding a first portion and a second portion; molding a guiding recess on a first surface of the first portion; molding a lubricating recess inside the guiding recess; applying a lubricant to the lubricating recess; engaging the first surface against a second surface of the second portion; and compressing the first and second portions together to form a seal by applying forces perpendicular to the first and second surfaces.

Clause 16. The method of clause 15, wherein compressing the first and second portions together comprises preventing fluid from passing between the first and second portions.

Clause 17. The method of clause 15, wherein compressing the first and second portions together comprises forming an insertion area that is configured to receive a center post that penetrates the seal and provides a fluid path through the seal.

Clause 18. The method of clause 15, wherein applying the lubricant to the lubricating recess assists the center post in penetrating the seal.

Clause 19. The method of clause 15, wherein a second guiding recess is molded on the second surface, a second lubricating recess is molded inside the second guiding recess, and the lubricant is applied to the second lubricating recess.

Clause 20. The method of clause 15, wherein at least one of the first and second surfaces comprise protrusions and intrusions, and the intrusions on the at least one of the first and second surfaces are configured to receive corresponding protrusions on the other of the at least one of the first and second surfaces, the method further comprising engaging the first surface against the second surface and mating each protrusion to its corresponding intrusion.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A seal for a needleless fluid connector, the seal comprising:
a first portion comprising a first top end, a first bottom end, and a first surface extending between the first top end and the first bottom end, and a second portion comprising a second top end, a second bottom end, and a second surface extending between the second top end and the second bottom end, the first portion being configured to couple with the second portion such that the first surface contacts the second surface and a longitudinal axis extends along the first and second surfaces from the first and second top ends to the first and second bottom ends;
a guiding recess and a lubricating recess on the first surface, the guiding recess extending from the first top end to at least the lubricating recess, and the lubricating recess positioned within the guiding recess and containing a lubricant,
wherein coupling of the first portion with the second portion forms an insertion area on the first top end aligned with the guiding recess.

2. The seal of claim 1, wherein the insertion area comprises an area of decreased tension that is formed from the guiding recess by compressing the first portion and the second portion together.

3. The seal of claim 1, wherein a fluid cannot permeate the insertion area and contact the first and second surfaces.

4. The seal of claim 1, wherein the insertion area assists a center post in penetrating the seal, and the center post creates a fluid path through the seal when the center post penetrates the seal.

5. The seal of claim 1, wherein the guiding recess is located along the longitudinal axis.

6. The seal of claim 1, wherein the guiding recess extends from the first top end to the first bottom end.

7. The seal of claim 1, wherein the lubricant in the lubricating recess is configured to lubricate the center post when the center post is inserted past the lubricating recess.

8. The seal of claim 1, wherein the second surface comprises a second guiding recess and a second lubricating recess, wherein the second guiding recess extends from the second top end to at least the second lubricating recess, and the second lubricating recess is positioned within the second guiding recess and contains the lubricant.

9. The seal of claim 8, wherein the second guiding recess is located along the longitudinal axis.

10. The seal of claim 8, wherein the second guiding recess extends from the second top end to the second bottom end.

11. The seal of claim 8, wherein the lubricant in the second lubricating recess is configured to lubricate the center post when the center post is inserted past the second lubricating recess.

12. The seal of claim 1, further comprising a divot located on at least one of the first and second bottom ends, wherein a path length between the first top end and the divot is less than a length between the first top end and the first bottom end, whereby the divot provides a shortened path of the center post through the seal.

13. The seal of claim 1, wherein the first and second surfaces each comprise protrusions and intrusions, and the protrusions and intrusions on the first surface correspond with intrusions and protrusions on the second surface.

14. The seal of claim 13, wherein each protrusion mates with its corresponding intrusion when the first portion and the second portion are compressed together.

15. A method for manufacturing a seal for a needleless medical fluid connector, the method comprising:
molding a first portion and a second portion;
molding a guiding recess on a first surface of the first portion;
molding a lubricating recess inside the guiding recess;
applying a lubricant to the lubricating recess;
engaging the first surface against a second surface of the second portion; and
compressing the first and second portions together to form a seal by applying forces perpendicular to the first and second surfaces.

16. The method of claim 15, wherein compressing the first and second portions together comprises preventing fluid from passing between the first and second portions.

17. The method of claim 15, wherein compressing the first and second portions together comprises forming an insertion area that is configured to receive a center post that penetrates the seal and provides a fluid path through the seal.

18. The method of claim 15, wherein applying the lubricant to the lubricating recess assists the center post in penetrating the seal.

19. The method of claim 15, wherein a second guiding recess is molded on the second surface, a second lubricating recess is molded inside the second guiding recess, and the lubricant is applied to the second lubricating recess.

20. The method of claim 15, wherein at least one of the first and second surfaces comprise protrusions and intrusions, and the intrusions on the at least one of the first and second surfaces are configured to receive corresponding protrusions on the other of the at least one of the first and second surfaces, the method further comprising engaging the first surface against the second surface and mating each protrusion to its corresponding intrusion.
